(19) United States
(12) Patent Application Publication
(10) Pub. No.: US 2025/0302718 A1
(43) Pub. Date: Oct. 2, 2025

(54) PHOTOCURABLE ARTIFICIAL NAIL COMPOSITION, ARTIFICIAL NAIL, AND METHOD FOR PRODUCING SAME

(71) Applicant: SHOFU INC., Higashiyama-ku (JP)

(72) Inventors: Yuko NAKASHOJI, Kyoto-shi (JP); Shun SHIMOSOYAMA, Kyoto-shi (JP)

(73) Assignee: SHOFU INC., Higashiyama-ku (JP)

(21) Appl. No.: 19/087,057

(22) Filed: Mar. 21, 2025

(30) Foreign Application Priority Data
Mar. 29, 2024 (JP) 2024-056149
Feb. 17, 2025 (JP) 2025-023342

(51) Int. Cl. A61K 8/55; A45D 31/00; A61K 8/37; A61K 8/87; A61Q 3/02

(52) U.S. Cl. CPC A61K 8/55; A45D 31/00; A61K 8/37; A61K 8/87; A61Q 3/02; A45D 2200/205; A61K 2800/30; A61K 2800/42; A61K 2800/81; A61K 2800/95

(57) ABSTRACT

It is an object of the present invention to provide a photocurable artificial nail composition that suppresses heat pain during curing, suppresses yellowing (including yellowing before curing), and has color tone stability. The photocurable artificial nail composition of the present invention includes a photopolymerization initiator and a polymerizable compound, in which the photopolymerization initiator contains a compound represented by General Formula (1), provided that the photopolymerization initiator does not contain an α-hydroxyalkylphenone photopolymerization initiator, and the composition does not contain a peroxide thermal polymerization initiator,

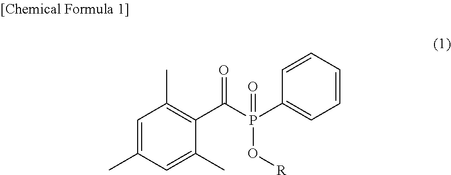

wherein R represents a hydrocarbon group having 1 to 5 carbon atoms.

### FIELD OF INVENTION

The present invention relates to a photocurable artificial nail composition that suppresses heat pain during curing, suppresses yellowing, and has color tone stability.

### BACKGROUND

Among artificial nails such as nail polishes, gel nails, and acrylic nails, gel nails are widely used from the viewpoints of good handleability, long-term lasting properties on a nail or an artificial nail, and low odor. The gel nails are photocurable artificial nail compositions that are applied onto a nail using a brush or the like to coat the nail, and cured with ultraviolet rays or visible rays.

While the gel nails are required to have long-term physical strength, handleability, and the like, they are required to shorten light irradiation time, and recently, a light irradiation device to which a low-illuminance fluorescent tube lamp is applied has been replaced by a high-illuminance visible light LED light. When a high-illuminance visible light LED light is used as the light irradiation device for gel nails, the curing time of the gel nails is shortened, and thus the treatment time is shortened. On the other hand, heat pain that the subject feels due to the curing heat (polymerization heat) generated at the time of photocuring is a problem.

JP 2019-6689A describes that a photocurable artificial nail composition containing a urethane (meth)acrylate oligomer, a polyfunctional thiol compound, a radically polymerizable compound, a photopolymerization initiator, and a chain transfer agent can suppress heat pain during curing.

JP 2023-170592A describes that a photocurable artificial nail composition containing a (meth)alkyl ester having a linear or branched alkyl group with 8 to 30 carbon atoms, a radically polymerizable component containing a compound having at least one radically polymerizable unsaturated double bond in the molecule, and a polymerization initiator can adjust a temperature rise during curing.

However, in particular, in a case where the application amount of gel nail is large, in a case where three-dimensional art is created, or the like, the coating film becomes thick, the subject is likely to feel heat pain, and there is room for improvement in the prior art.

In addition, gel nails are required to have an aesthetic appearance, but there is a problem that yellowing due to a photopolymerization initiator occurs during curing. Also, there is a case where the gel itself exhibits a light yellow color, and thus there is a problem that there is a case where the aesthetic appearance of the gel itself before curing is poor. As disclosed in JP 2022-56207A, yellowing (including coloring into yellow before curing) is conventionally suppressed by blending a predetermined amount of a violet/blue coloring matter that is a complementary color of yellow. However, the yellowing has not been fundamentally solved.

### SUMMARY OF INVENTION

It is an object of the present invention to provide a photocurable artificial nail composition that suppresses heat pain during curing, suppresses yellowing (including yellowing before curing), and has color tone stability.

Means for solving the above problems includes the following aspects.

<1>

A photocurable artificial nail composition including a photopolymerization initiator and a polymerizable compound,
in which the photopolymerization initiator contains a compound represented by General Formula (1), provided that the photopolymerization initiator does not contain an α-hydroxyalkylphenone photopolymerization initiator, and
the photocurable artificial nail composition does not contain a peroxide thermal polymerization initiator,

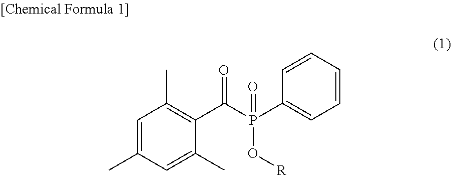

wherein R represents a hydrocarbon group having 1 to 5 carbon atoms.
   <2>

The photocurable artificial nail composition according to <1>, in which the photopolymerization initiator contains only the compound represented by General Formula (1).

<3>

The photocurable artificial nail composition according to <1>, in which the photopolymerization initiator further contains 2,4,6-trimethylbenzoyldiphenylphosphine oxide and/or bis(2,4,6-trimethylbenzoyl)phenylphosphine oxide.

<4>

The photocurable artificial nail composition according to any one of <1> to <3>, in which the polymerizable compound contains at least one selected from the group consisting of an oligomer and a polymerizable monomer.

<5>

The photocurable artificial nail composition according to any one of <1> to <3>, in which the polymerizable compound contains an oligomer and a polymerizable monomer.

<6>

The photocurable artificial nail composition according to <4> or <5>, in which the oligomer contains a urethane di(meth)acrylate oligomer.

<7>

The photocurable artificial nail composition according to any one of <1> to <6>, containing 0.1 to 10 mass % of the compound represented by General Formula (1).

<8>

The photocurable artificial nail composition according to any one of <1> to <7>, containing 20 mass % or more of the polymerizable compound.

<9>

The photocurable artificial nail composition according to any one of <5> to <8>, in which a content of the oligomer is 50 to 750 parts by mass per 100 parts by mass of the polymerizable monomer.

<10>

The photocurable artificial nail composition according to any one of <1> to <9>, which has a viscosity of 1 to 2,000,000 mPa·s.

<11>

An artificial nail obtained from the photocurable artificial nail composition according to any one of <1> to <10>.

<12>

A method for producing an artificial nail, including the step of applying the photocurable artificial nail composition according to any one of <1> to <10> onto a nail or a substrate and curing the photocurable artificial nail composition.

<13>

A method for forming an artificial nail, including applying a photocurable artificial nail composition that contains a photopolymerization initiator and a polymerizable compound onto a nail or a substrate and curing the photocurable artificial nail composition,
in which the photopolymerization initiator contains a compound represented by General Formula (1), provided that the photopolymerization initiator does not contain an α-hydroxyalkylphenone photopolymerization initiator, and
the photocurable artificial nail composition does not contain a peroxide thermal polymerization initiator,

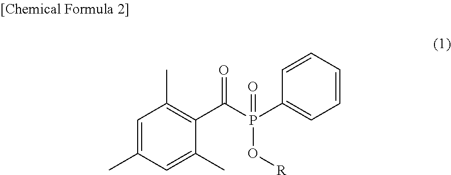

wherein R represents a hydrocarbon group having 1 to 5 carbon atoms.
   <14>

Use of a compound represented by General Formula (1) for producing a photocurable artificial nail composition that contains a photopolymerization initiator (provided that the photopolymerization initiator does not contain an α-hydroxyalkylphenone photopolymerization initiator) and a polymerizable compound, and does not contain peroxide thermal polymerization initiator,

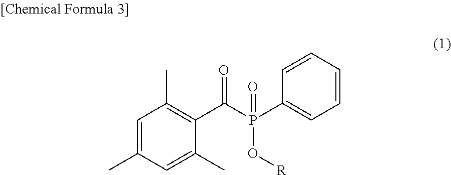

wherein R represents a hydrocarbon group having 1 to 5 carbon atoms.

According to the present invention, it is possible to provide a photocurable artificial nail composition that suppresses heat pain during curing, suppresses yellowing (including yellowing before curing), and has color tone stability.

### BRIEF DESCRIPTION OF DRAWINGS

FIG. 1 shows photographs of yellowness test specimens in the examples and the comparative examples (from the left, Example 6, Comparative Example 1, and Comparative Example 2).

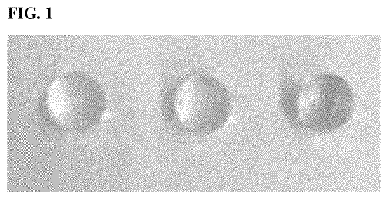

### DETAILED DESCRIPTION OF THE PREFERRED EMBODIMENTS

### 1. Photocurable Artificial Nail Composition

The present invention can provide a photocurable artificial nail composition that suppresses heat pain during curing, suppresses yellowing (including coloring into yellow before curing), and has color tone stability.

The photocurable artificial nail composition of the present invention includes a photopolymerization initiator and a polymerizable compound, and the photopolymerization initiator contains a compound represented by General Formula (1), provided that the photopolymerization initiator does not contain an α-hydroxyalkylphenone photopolymerization initiator, and the composition does not contain a peroxide thermal polymerization initiator. Hereinafter, the photocurable artificial nail composition of the present invention is described in detail.

### [Photopolymerization Initiator]

In the present invention, the photopolymerization initiator contains a compound represented by General Formula (1).

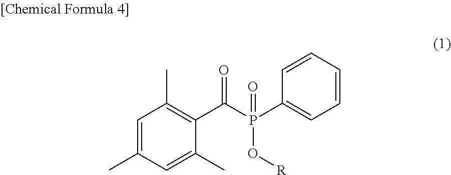

In General Formula (1), R represents a hydrocarbon group having 1 to 5 carbon atoms. The structure of the hydrocarbon group having 1 to 5 carbon atoms is not particularly limited, and may be any of a linear structure, a branched structure, a cyclic structure, or the like. Specific examples of R include aliphatic hydrocarbon groups, for example, alkyl groups such as a methyl group, an ethyl group, a propyl group, an isopropyl group, a cyclopropyl group, a butyl group, an isobutyl group, a cyclobutyl group, a cyclopropylmethyl group, a pentyl group, an isopentyl group, a cyclopentyl group, a cyclopropylethyl group, a cyclopropyldimethyl group, and a cyclobutylmethyl group, alkenyl groups such as a vinyl group, a propenyl group, a butenyl group, and a pentenyl group, and alkynyl groups such as an ethynyl group, a propynyl group, a butynyl group, and a pentynyl group. Among them, an alkyl group is preferable, an ethyl group or a propyl group is more preferable, and an ethyl group is particularly preferable.

The compound represented by General Formula (1) is a compound that acts as a radical polymerization initiator. Since the photocurable artificial nail composition of the present invention contains the compound represented by General Formula (1) as a radical polymerization initiator, a rapid temperature rise is less likely to occur during curing, the heat pain of the subject can be suppressed, and in addition, the photocurable artificial nail composition is less likely to exhibit yellow color before curing, and is less likely to cause yellowing even after curing.

The content of the compound represented by General Formula (1) in the photocurable artificial nail composition of the present invention is not particularly limited, but is preferably in the range of 0.1 to 10 mass %, more preferably in the range of 0.8 to 8.0 mass % from the viewpoint that properties such as curability, surface hardness, strength, and durability are further improved, still more preferably in the range of 1.6 to 8.0 mass % from the viewpoint that properties such as curability, surface hardness, strength, durability, and surface curability are further improved, even still more preferably in the range of 2.0 to 6.0 mass % from the viewpoint that in addition to properties such as curability, surface hardness, strength, durability, and surface curability, the effects of heat pain suppression and color tone stability of the present invention are further enhanced, and particularly preferably in the range of 2.4 to 6.0 mass % from the viewpoint that in addition to properties such as curability, surface hardness, strength, durability, and surface curability, the effects of heat pain suppression and color tone stability of the present invention are still further enhanced. When the content of the compound represented by General Formula (1) is within the above range, the effects of heat pain suppression and color tone stability of the present invention can be further enhanced.

From the viewpoint that the effect of the present invention is improved, the photopolymerization initiator preferably contains only the compound represented by General Formula (1), but may contain 2,4,6-trimethylbenzoyldiphenylphosphine oxide and/or bis(2,4,6-trimethylbenzoyl)phenylphosphine oxide together with the compound represented by General Formula (1). When the photopolymerization initiator contains 2,4,6-trimethylbenzoyldiphenylphosphine oxide and/or bis(2,4,6-trimethylbenzoyl)phenylphosphine oxide, the content thereof (the total content when two are contained) is, for example, 0.1 to 5 parts by mass per part by mass of the compound represented by General Formula (1), and from the viewpoint that the effect of the present invention is improved, the content is preferably 0.1 to 4 parts by mass, more preferably 0.1 to 3 parts by mass, still more preferably 0.1 to 2 parts by mass, and even still more preferably 0.1 to 1 part by mass.

The photopolymerization initiator may contain other photopolymerization initiators other than the above compounds, specifically, a radical polymerization initiator that generates a radical by irradiation with an energy ray such as visible light, ultraviolet light, X-ray, or electron beam, a cationic polymerization initiator that generates a cation, and an anionic polymerization initiator that generates an anion. These photopolymerization initiators may be used singly or in combination of two or more kinds thereof. Among them, a radical polymerization initiator is preferable. Examples of the radical photopolymerization initiator include benzoin ethers, benzyl ketals, α-dialkoxyacetophenones, α-aminoalkylphenones, acylphosphine oxides, benzophenones, thioxanthones, and titanocenes. These radical photopolymerization initiators may be used singly or in combination of two or more kinds thereof.

When the photopolymerization initiator contains other photopolymerization initiators other than the compound represented by General Formula (1), the total content of the other photopolymerization initiators is preferably 80 mass % or less, more preferably 75 mass % or less, still more preferably 50 mass % or less, and particularly preferably 25 mass % or less with respect to the total amount of the photopolymerization initiators. When the total content of the other photopolymerization initiators is within the above range, the effects of heat pain suppression and color tone stability of the present invention can be further enhanced.

However, in the present invention, the photopolymerization initiator does not contain an α-hydroxyalkylphenone photopolymerization initiator in order to obtain the effects of heat pain suppression and color tone stability of the present invention.

Examples of the α-hydroxyalkylphenone photopolymerization initiator include 1-hydroxy-cyclohexylphenyl ketone, 2-hydroxy-2-methyl-1-phenyl-propane-1-one, 1-[4-(2-hydroxymethoxy)-phenyl]-2-hydroxy-2-methyl-1-propane-1-one, 2-hydroxy-1-[4-[4-(2-hydroxy-2-methyl-propionyl)-benzyl]phenyl]-2-methyl-propane-1-one, 1-(4-(phenylthio)-2,2-(O-benzoyloxime)) 1-hydroxycyclohexylphenyl ketone, and 1-(4-isopropylphenyl)-2-hydroxy-2-methylpropane-1-one.

In order to obtain the effects of heat pain suppression and color tone stability of the present invention, the photocurable artificial nail composition of the present invention does not contain a peroxide thermal polymerization initiator. In order to improve the effects of heat pain suppression and color tone stability of the present invention, the photocurable artificial nail composition of the present invention preferably does not contain a thermal polymerization initiator.

Examples of the peroxide thermal polymerization initiator include t-butyl hydroperoxide, cumene hydroperoxide, diacetyl peroxide, didecanoyl peroxide, di-t-butyl peroxide, methyl ethyl ketone peroxide, cyclohexanone peroxide, dicumyl peroxide, di(3,5,5-trimethylhexanoyl) peroxide, 2,5-dimethylhexane-2,5-dihydroperoxide, dilauroyl peroxide, disuccinic acid peroxide, dibenzoyl peroxide, parachlorobenzoyl peroxide, 2,4-dichlorobenzoyl peroxide, t-butyl peroxypivalate, t-butyl peroxybenzoate, t-hexyl peroxide valerate, 2,5-dimethyl-2,5-di(2-ethylhexanoylperoxy) hexane, 1,1,3,3-tetramethylbutyl peroxy-2-ethylhexanoate, t-hexyl peroxy-2-ethylhexanoate, t-butyl peroxy-2-ethylhexanoate, n-butyl-4,4-di(t-butylperoxy) valerate, 1,1-di(t-butylperoxy) cyclohexane, 1,1-di(t-hexylperoxy) cyclohexane, 2,2-bis(4,4-di-t-butylperoxycyclohexyl) propane, 1,1-bis(t-butylperoxy)-3,3,5-trimethylcyclohexane, 2,2-bis(t-butylperoxy) butane, 2,2-bis(t-butylperoxy) octane, dicetyl peroxydicarbonate, t-hexyl peroxy isopropyl monocarbonate, diisopropyl peroxydicarbonate, t-butyl peroxy isopropyl carbonate, di(4-t-butylcyclohexyl) peroxydicarbonate, and di(2-ethylhexyl) peroxydicarbonate.

### [Polymerizable Compound]

The polymerizable compound contained in the photocurable artificial nail composition of the present invention is a substance that reacts with light or heat to form a polymer. The polymerizable compound imparts properties such as curability, surface hardness, strength, flexibility, durability, and removability to the photocurable artificial nail composition of the present invention. The polymerizable compound is not particularly limited as long as it is a compound (for example, a polymerizable monomer, an oligomer, or a polymer) having at least one ethylenically unsaturated group as a polymerizable functional group, and a known polymerizable compound can be used. Specific examples of the ethylenically unsaturated group include a (meth)acryloyl group, a (meth)acryloyloxy group, a (meth)acrylamide group, a vinyl group, a vinyl ether group, a methyl vinyl ether group, an allyl group, an allyl ether group, and a maleimide group, but are not limited thereto. One kind of the ethylenically unsaturated group may be contained, or two or more kinds thereof may be contained. Among them, from the viewpoint of curability, surface hardness, and durability, at least one selected from the group consisting of a (meth)acryloyl group and a (meth)acryloyloxy group is preferable.

In the present specification, the term “(meth)acryloyl” encompasses both acryloyl and methacryloyl, the term “(meth)acrylate” encompasses both acrylate and methacrylate, the term “(meth)acryloyloxy” encompasses both acryloyloxy and methacryloyloxy, and the term “(meth)acrylamide” encompasses both acrylamide and methacrylamide.

Examples of the polymerizable compound having one ethylenically unsaturated group in one molecule include polymerizable monomers such as methacrylic acid, acrylic acid, urethane (meth)acrylate, methoxyethylene glycol (meth)acrylate, methoxypolyethylene glycol (meth)acrylate, phenoxyethylene glycol (meth)acrylate, phenoxypolyethylene glycol (meth)acrylate, 2-hydroxy-3-phenoxypropyl (meth)acrylate, 2-(meth)acryloyloxyethyl succinate, 2-(meth)acryloyloxyethyl phthalate, 2-(meth)acryloyloxypropyl hexaphthalate, stearyl (meth)acrylate, methyl (meth)acrylate, ethyl (meth)acrylate, hydroxyethyl (meth)acrylate, hydroxypropyl (meth)acrylate, 3-chloro-2-hydroxypropyl (meth)acrylate, isobornyl (meth)acrylate, tetrahydrofurfuryl (meth)acrylate, 2-(meth)acryloyloxyethyl dihydrogen phosphate, 2-(meth)acryloyloxyethyl phenyl hydrogen phosphate, 10-(meth)acryloyloxydecyl dihydrogen phosphate, 6-(meth)acryloyloxyhexyl dihydrogen phosphate, 2-(meth)acryloyloxyethyl 2-bromoethyl hydrogen phosphate, methylol (meth)acrylamide, dimethyl (meth)acrylamide, (meth)acryloyl morpholine, and vinyl chloride; oligomers of the polymerizable monomers; and polymers of the polymerizable monomers, but are not limited thereto. The oligomer and the polymer may contain one or two or more of the polymerizable monomers exemplified above.

In the present specification, the term “oligomer” means a polymer obtained by polymerizing 2 to several tens of polymerizable monomers, and the term “polymer” means a polymer other than an oligomer, obtained by polymerizing several tens or more of polymerizable monomers.

Examples of the polymerizable compound having two ethylenically unsaturated groups in one molecule include polymerizable monomers such as urethane di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, glycerin di(meth)acrylate, ethylene glycol di(meth)acrylate, triethylene glycol di(meth)acrylate, polyethylene glycol di(meth)acrylate, propylene glycol di(meth)acrylate, polypropylene glycol di(meth)acrylate, ethoxylated polypropylene glycol di(meth)acrylate, ethoxylated propylene glycol di(meth)acrylate, ethoxylated bisphenol A di(meth)acrylate, propoxylated bisphenol A di(meth)acrylate, propoxylated ethoxylated bisphenol A di(meth)acrylate, tricyclodecanedimethanol di(meth)acrylate, and bis[2-(methacryloyloxy)ethyl] phosphate; oligomers of the polymerizable monomers; and polymers of the polymerizable monomers, but are not limited thereto. The oligomer and the polymer may contain one or two or more of the polymerizable monomers exemplified above.

Examples of the polymerizable compound having three or more ethylenically unsaturated groups in one molecule include polymerizable monomers such as urethane tri(meth)acrylate, urethane tetra(meth)acrylate, urethane penta(meth)acrylate, urethane hexa(meth)acrylate, trimethylolpropane tri(meth)acrylate, ethoxylated trimethylolpropane tri(meth)acrylate, ethoxylated glycerin tri(meth)acrylate, ditrimethylolpropane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, ethoxylated isocyanurate tri(meth)acrylate, pentaerythritol tetra(meth)acrylate, tetramethylolmethane tetra(meth)acrylate, dipentaerythritol tetra(meth)acrylate, ditrimethylolpropane tetra(meth)acrylate, dipentaerythritol penta(meth)acrylate, caprolactone-modified pentaerythritol penta(meth)acrylate, dipentaerythritol hexa(meth)acrylate, and caprolactone-modified pentaerythritol hexa(meth)acrylate; oligomers of the polymerizable monomers; and polymers of the polymerizable monomers, but are not limited thereto. The oligomer and the polymer may contain one or two or more of the polymerizable monomers exemplified above.

The molecular weight of the polymerizable monomer is not particularly limited, but is preferably less than 1,000. The weight average molecular weight (Mw) of the oligomer is not particularly limited, but is, for example, 200 to 100,000, preferably 200 to 50,000, more preferably 300 to 30,000, and particularly preferably 700 to 10,000. Within this range, properties such as curability, surface hardness, strength, flexibility, durability, and removability can be improved. In the present specification, as the weight average molecular weight (Mw), a value measured by gel permeation chromatography (GPC) using polystyrene as a standard substance, is adopted.

The polymerizable monomer is preferably a liquid and has fluidity in an atmosphere at 23° C. Specifically, the polymerizable monomer preferably has a viscosity of 20,000 mPa's or less, more preferably 10,000 mPa's or less, still more preferably 5,000 mPa's or less, and particularly preferably 1,000 mPa's or less at 23° C. and a shear rate of 10 s⁻¹ as measured using a rheometer, which is a dynamic viscoelasticity measuring device. The oligomer may or may not have fluidity in an atmosphere at 23° C. Specifically, the oligomer preferably has a viscosity of 9,000 mPa's or more, more preferably 9,000 to 3,000,000 mPa s, still more preferably 9,000 to 2,500,000 mPa s, and particularly preferably 9,000 to 2,200,000 mPa's at 23° C. and a shear rate of 10 s⁻¹ as measured using a rheometer, which is a dynamic viscoelasticity measuring device. The polymer may or may not have fluidity in an atmosphere at 23° C.

Two or more polymerizable compounds can be selected to form a photocurable artificial nail composition. Among these examples, it is preferable to use at least one selected from the group consisting of methacrylic acid, acrylic acid, urethane (meth)acrylate, urethane di(meth)acrylate, urethane tri(meth)acrylate, urethane tetra(meth)acrylate, urethane penta(meth)acrylate, urethane hexa(meth)acrylate, isobornyl (meth)acrylate, tetrahydrofurfuryl (meth)acrylate, hydroxypropyl (meth)acrylate, triethylene glycol di(meth)acrylate, (meth)acryloylmorpholine, bis[2-(methacryloyloxy)ethyl] phosphate, trimethylolpropane tri(meth)acrylate, oligomers thereof, and polymers thereof. The oligomer and the polymer may contain one or two or more of the polymerizable monomers exemplified above as the constituent unit. The polymerizable compound more preferably contains the oligomer and the polymerizable monomer, still more preferably contains at least one oligomer selected from the group consisting of a urethane (meth)acrylate oligomer, a urethane di(meth)acrylate oligomer, and a urethane hexa(meth)acrylate oligomer and at least one polymerizable monomer selected from the group consisting of isobornyl (meth)acrylate, tetrahydrofurfuryl (meth)acrylate, hydroxypropyl (meth)acrylate, triethylene glycol di(meth)acrylate, (meth)acryloylmorpholine, and trimethylolpropane tri(meth)acrylate, and particularly preferably contains a urethane di(meth)acrylate oligomer and at least one polymerizable monomer selected from the group consisting of isobornyl (meth)acrylate, tetrahydrofurfuryl (meth)acrylate, and trimethylolpropane tri(meth)acrylate. When the polymerizable compound contains the oligomer and the polymerizable monomer, properties such as flexibility, durability, removability, curability, surface hardness, and strength can be improved.

The content of the polymerizable compound is not particularly limited, but the content of the polymerizable compound with respect to the total amount of the composition is preferably 20 mass % or more, more preferably within the range of 30 to 99.2 mass %, still more preferably within the range of 30 to 98.4 mass %, even still more preferably within the range of 40 to 97.6 mass %, further preferably within the range of 60 to 96 mass %, and particularly preferably within the range of 80 to 96 mass %. When the content of the polymerizable compound is within the above range, properties such as flexibility, durability, removability, curability, surface hardness, and strength can be improved.

When the polymerizable compound contains an oligomer and a polymerizable monomer, the content of the oligomer is usually about 10 to 1000 parts by mass per 100 parts by mass of the polymerizable monomer, and is preferably 50 to 750 parts by mass from the viewpoint that properties such as flexibility, durability, removability, curability, surface hardness, and strength are further improved, and it is more preferably 100 to 350 parts by mass, still more preferably 150 to 300 parts by mass, and particularly preferably 150 to 250 parts by mass from the viewpoint that in addition to properties such as flexibility, durability, removability, curability, surface hardness, and hardness, the effects of heat pain suppression and color tone stability of the present invention are further enhanced. When the photocurable artificial nail composition contains a polymerizable curing accelerator among the curing accelerators described later, the preferred content of the oligomer is calculated as the content with respect to the total of the polymerizable monomer and the curing accelerator. The heat pain due to the curing heat tends to appear more remarkably as the coating film is thicker and the ethylenically unsaturated group equivalent is larger. When the content of the oligomer with respect to the total amount of the polymerizable compound increases, the viscosity of the photocurable artificial nail composition increases and thus the coating film becomes thick, but the ethylenically unsaturated group equivalent decreases. On the other hand, when the content of the polymerizable monomer with respect to the total amount of the polymerizable compound increases, the viscosity of the photocurable artificial nail composition decreases and thus the coating film becomes thin, but the ethylenically unsaturated group equivalent increases. When the content of the oligomer per 100 parts by mass of the polymerizable monomer is within the above range, the effect of heat pain suppression of the present invention can be further enhanced.

### [Other Components]

The photocurable artificial nail composition of the present invention may contain other components other than the photopolymerization initiator or the polymerizable compound as long as the effect of the present invention is not impaired. Examples of other components include solvents, auxiliary agents, additives, colorants, leveling agents, plasticizers, antioxidants, thermal polymerization initiators that initiate polymerization reactions by heating to a certain temperature or higher (provided that they are other than peroxide thermal polymerization initiators), chemical polymerization initiators that initiate polymerization reactions by mixing specific substances and are types in which two or more agent are mixed, polymerization accelerators, polymerization inhibitors, coalescing agents, preservatives, waxes, thickeners, fragrances, UV shielding agents, diffusing agents, defoamers, dispersants, fillers, surfactants, pigments, dyes, excipients, ion releasing agents, and silane coupling agents, which are widely used in photocurable artificial nail compositions. These may be used singly or in combination of two or more kinds thereof.

As the polymerization accelerator, a polyfunctional thiol compound having two or more thiol groups in one molecule can be used. When the polyfunctional thiol compound is used, the curing reaction proceeds without being inhibited by oxygen. Examples of specific compounds as the polyfunctional thiol compound include 1,2-ethanedithiol, 1,2-propanedithiol, 1,3-propanedithiol, 1,3-butanedithiol, 2,3-butanedithiol, 1,5-pentanedithiol, 1,6-hexanedithiol, 1,8-octanedithiol, 1,9-nonanedithiol, 1,10-decanedithiol, 1,2-benzenedithiol, 1,3-benzenedithiol, 1,4-benzenedithiol, 3,6-dichloro-1,2-benzenedithiol, toluene-3,4-dithiol, 1,5-naphthalenedithiol, ethylene glycol bis(thioglycolate), ethylene glycol bis(3-mercaptopropionate), 1,4-butanediol bisthioglycolate, tetraethylene glycol bis(3-mercaptopropionate), trimethylolpropane tris(thioglycolate), trimethylolpropane tris(3-mercaptopropionate), trimethylolpropane tris(3-mercaptobutyrate), tris[(3-mercaptopropionyloxy)-ethyl] isocyanurate, pentaerythritol tetrakis(thioglycolate), pentaerythritol tetrakis(3-mercaptopropionate), dipentaerythritol hexakis (3-mercaptopropionate), 1,4-bis(3-mercaptobutyryloxy) butane, pentaerythritol tetrakis(3-mercaptobutyrate), 1,3,5-tris(3-mercaptobutyloxyethyl)-1,3,5-triazine-2,4,6 (1H,3H,5H)-trione, dimercaptodiethyl sulfide, 1,8-dimercapto-3,6-dithiaoctane, 1,2-bis[(2-mercaptoethyl)thio]-3-mercaptopropane, tetrakis(7-mercapto-2,5-dithiaheptyl) methane, trithiocyanuric acid, 1,2-benzenedimethanethiol, 4,4′-thiobisbenzenethiol, 2-di-n-butylamino-4,6-dimercapto-s-triazine, 2,5-dimercapto-1,3,4-thiadiazole, 1,8-dimercapto-3,6-dioxaoctane, 1,5-dimercapto-3-thiapentane, tris(2-hydroxyethyl) isocyanurate trimercaptopropionate, 1,4-dimethylmercaptobenzene, 2,4,6-trimercapto-s-triazine, 2-(N,N-dibutylamino)-4,6-dimercapto-s-triazine, bis(4-(2-mercaptopropoxy)phenyl) methane, 1,1-bis(4-(2-mercaptopropoxy)phenyl) ethane, 2,2-bis(4-(2-mercaptopropoxy)phenyl) propane, 2,2-bis(4-(2-mercaptopropoxy)phenyl) butane, 1,1-bis(4-(2-mercaptopropoxy)phenyl) isobutane, 2,2-bis(4-(2-mercaptopropoxy)-3-methylphenyl) propane, 2,2-bis(4-(2-mercaptopropoxy)-5-methylphenyl) propane, bis(2-(2-mercaptopropoxy)-5-methylphenyl) methane, 2,2-bis(4-(2-mercaptopropoxy)-3-t-butylphenyl) propane, tris(4-(2-mercaptopropoxy)phenyl) methane, 1,1,1-tris(4-(2-mercaptopropoxy)phenyl) ethane, bis(4-(2-mercaptobutoxy)phenyl) methane, 2,2-bis(4-(2-mercaptobutoxy)phenyl) propane, tris(4-(2-mercaptobutoxy)phenyl) methane, 1,3,5-triazine-2,4,6-trithiol, and alkyl vinyl ether adducts thereof. However, the polymerization accelerator is not limited thereto. These polymerization accelerators may be used singly or in combination of two or more kinds thereof.

As the polymerization accelerator, tertiary amines can also be used other than the polyfunctional thiol compound. Specific examples of the tertiary amines include N,N-dimethylaniline, N,N-diethylaniline, N,N-di-n-butylaniline, N,N-dibenzylaniline, N,N-dimethyl-p-toluidine, N,N-dimethyl-m-toluidine, N,N-diethyl-p-toluidine, p-bromo-N,N-dimethylaniline, m-chloro-N,N-dimethylaniline, p-dimethylaminobenzaldehyde, p-dimethylaminoacetophenone, p-dimethylaminobenzoic acid, p-dimethylaminobenzoic acid ethyl ester, p-dimethylaminobenzoic acid amino ester, N,N-dimethylanthranilic acid methyl ester, N,N-dihydroxyethylaniline, N,N-dihydroxyethyl-p-toluidine, p-dimethylaminophenyl alcohol, p-dimethylaminostyrene, N,N-dimethyl-3,5-xylidine, 4-dimethylaminopyridine, N,N-dimethyl-α-naphthylamine, N,N-dimethyl-β-naphthylamine, tributylamine, tripropylamine, triethylamine, N-methyldiethanolamine, N-ethyldiethanolamine, N,N-dimethylhexylamine, N,N-dimethyldodecylamine, N,N-dimethylstearylamine, N,N-dimethylaminoethyl methacrylate, N,N-diethylaminoethyl methacrylate, and 2,2′-(n-butylimino) diethanol. However, the polymerization accelerator is not limited thereto. These polymerization accelerators may be used singly or in combination of two or more kinds thereof.

Some polymerization accelerators cause a polymerization reaction with a polymerizable compound. Such a polymerization accelerator is regarded as a polymerizable compound even when it does not have an ethylenically unsaturated group in its structure. Thus, in the calculation of the preferred content of the oligomer when the polymerizable compound contains the oligomer and the polymerizable monomer, 100 parts by weight of the polymerizable monomer means the total amount of the polymerizable monomer and the polymerization accelerator having polymerizability, and the content is calculated based on this.

When the polymerization accelerator is contained, the content thereof is not particularly limited, but is preferably 4 mass % or more, more preferably 6 mass % or more, still more preferably 11 mass % or more, and particularly preferably 15 mass % or more with respect to the total amount of the composition. The upper limit of the content is also not particularly limited, but is preferably 80 mass % or less, more preferably 70 mass % or less, still more preferably 60 mass % or less, and particularly preferably 30 mass % or less. When the content of the polymerization accelerator is within the above range, properties such as flexibility, durability, removability, curability, surface hardness, and strength are improved, and polymerization is not inhibited by oxygen, so that good surface curability can be exhibited.

### [Usage, Properties, Form, and Others of Photocurable Artificial Nail Composition]

The photocurable artificial nail composition of the present invention can be applied to a natural nail; a nail on which a coating film is formed by an artificial nail composition; and a substrate such as an artificial resin chip, a resin film, or a resin sheet, but the objects to which the composition can be applied are not limited thereto. Examples of the method of application include methods of application with a brush, a sponge, a spray, an inkjet, an air knife, a roll, or the like, but are not limited thereto.

The viscosity of the photocurable artificial nail composition of the present invention is not particularly limited, but from the viewpoint of handleability, the photocurable artificial nail composition preferably has fluidity in an atmosphere at 23° C. Specifically, the viscosity at 23° C. and a shear rate of 10 s⁻¹ measured using a rheometer, which is a dynamic viscoelasticity measuring device, is preferably 2,000,000 mPa's or less, more preferably 1,000,000 mPa's or less, still more preferably 500,000 mPa's or less, even still more preferably 200,000 mPa's or less, further preferably 100,000 mPas or less, and particularly preferably 50,000 mPa's or less. The lower limit of the viscosity is not particularly limited, but is preferably 1 mPa's or more, more preferably 500 mPa's or more, still more preferably 1,000 mPa's or more, and particularly preferably 5,000 mPa's or more. Specific examples of the viscosity of the photocurable artificial nail composition of the present invention include 1,000 to 2,000,000 mPas, 5,000 to 2,000,000 mPas, 5,000 to 1,000,000 mPas, 5,000 to 500,000 mPa s, and 5,000 to 200,000 mPa s. When the viscosity is less than 1 mPa's, since the viscosity is low, a phenomenon such as dropping of the applied photocurable artificial nail composition from the applied surface is observed, and a uniform applied surface cannot be formed, as a result of which the photocurable artificial nail composition has poor properties in terms of handleability. The heat pain due to the curing heat tends to appear more remarkably as the coating film is thicker, and the thickness of the coating film tends to increase as the viscosity of the photocurable artificial nail composition increases. When the viscosity of the photocurable artificial nail composition decreases, the coating film becomes thin, whereby heat pain is less likely to be felt. However, decreasing the viscosity may adversely affect handleability, for example, it causes flowing of the applied photocurable artificial nail composition to an unintended place. On the other hand, when the viscosity of the photocurable artificial nail composition increases, the coating film becomes thick, whereby heat pain is likely to be felt. However, increasing the viscosity may have a good influence on handleability, for example, it becomes possible to create three-dimensional art. When the viscosity of the photocurable artificial nail composition is within the above range, the heat pain suppressing effect of the present invention can be further enhanced while the photocurable artificial nail composition has good handleability according to various purposes. The viscosity of the photocurable artificial nail composition of the present invention can be changed by appropriately adjusting the kind and content of the polymerizable compound.

The form of the photocurable artificial nail composition of the present invention is not particularly limited, and may be a nail polish, a gel nail, an acrylic nail, or the like. Nail polishes are coating materials for nails called nail lacquer, nail enamel, manicure, or the like, and are compositions for producing coating films excellent in aesthetic appearance by drying a contained solvent. Gel nails are materials containing a resin component that is cured by ultraviolet rays or visible rays, and are compositions that are applied onto natural nails or artificial nails and then cured by irradiation with ultraviolet rays or visible rays to form coating films excellent in aesthetic appearance. Acrylic nails are powder-liquid type materials containing polymer beads and a polymerizable monomer, and are compositions which are cured by polymerization of the polymerizable monomer initiated by a polymerization initiator contained in the polymer beads after powder-liquid mixing. The acrylic nails are characterized in that they can not only be applied but also be produced in built-up forms, and are mainly used for extending nails in many cases. Among these, the photocurable artificial nail composition of the present invention is preferably provided in the form of a gel nail. In the case of a form of gel nail, it is possible to provide a photocurable artificial nail composition that suppresses heat pain during curing, suppresses yellowing (including yellowing before curing), and has color tone stability.

In a gel nail, a base layer, a color layer, and a topcoat layer are generally laminated in this order on a natural nail or a substrate to form a coating film. The application of the photocurable artificial nail composition of the present invention is not particularly limited, and the artificial nail composition may be any layer of a base layer, a color layer, or a topcoat layer. Since heat pain is likely to be felt when a gel nail is directly applied to a fingernail or a toenail, the photocurable artificial nail composition of the present invention is preferably used as a base layer. In addition, since gel nails are required to have curability, surface strength, and durability, the photocurable artificial nail composition of the present invention is more preferably used as a topcoat layer in which it is necessary to blend a large amounts of a component that tends to increase the curing heat or strongly exhibits a yellowness before and after curing.

Gel nails include those for hands to be applied to fingernails, those for feet to be applied to toenails, and those for animals to be applied to animal nails. The use of the photocurable artificial nail composition of the present invention is not particularly limited, and the photocurable artificial nail composition can be used for any of hands, feet, animals, or the like.

### 2. Artificial Nail and Method for Producing Same

The artificial nail of the present invention is obtained from the photocurable artificial nail composition of the present invention. The artificial nail of the present invention can be formed, for example, by applying the photocurable artificial nail composition of the present invention to a natural nail, a nail on which a coating film is formed by a photocurable artificial nail composition, or a substrate such as an artificial resin chip, a resin film, or a resin sheet, and curing the composition. The method of application is not particularly limited, and examples thereof include methods of application with a brush, a sponge, a spray, an inkjet, an air knife, a roll, or the like. The curing method is not particularly limited, and examples thereof include a method of curing by irradiation with ultraviolet rays or visible rays and a method of curing by heating, and from the viewpoint of rapid curing, a method of curing by irradiation with ultraviolet rays or visible rays is preferred.

### EXAMPLES

Hereinafter, the examples and the comparative examples of the present invention is specifically described, but the present invention is not limited to these examples.

### [Components Used for Preparation of Photocurable Artificial Nail Composition]

The components used for preparing the photocurable artificial nail compositions of the examples and the comparative examples are shown below. As the weight average molecular weight (Mw), a value measured by gel permeation chromatography (GPC) using a GPC analyzer (manufactured by Shimadzu Corporation, trade name: Nexera GPC system), a column (manufactured by Waters Corporation, trade name: Styragel HR), tetrahydrofuran as an eluent, and polystyrene as a standard substance, was adopted.

Abbreviations shown in Tables 1 to 5 are as follows.
A1: Urethane diacrylate oligomer (weight average molecular weight: 8400)
A2: Urethane diacrylate oligomer (weight average molecular weight: 740)
A3: Isobornyl methacrylate
A4: Trimethylolpropane triacrylate
A5: Acrylic acid
A6: Methacrylic acid
A7: Bis[2-(methacryloyloxy)ethyl] phosphate
Compound 1: (2,4,6-Trimethylbenzoyl)phenylethoxyphosphine oxide (compound of General Formula (1), wherein R is an ethyl group)
Compound 2:2,4,6-Trimethylbenzoyldiphenylphosphine oxide
Compound 3: Bis(2,4,6-trimethylbenzoyl)phenylphosphine oxide

### [Preparation of Photocurable Artificial Nail Composition]

The amount of each component was calculated according to each blending ratio described in Tables 1 to 5, and the components were mixed until a homogeneous liquid was obtained using a planetary centrifugal mixer (manufactured by THINKY CORPORATION, trade name: ARV-310P) under atmospheric pressure to prepare each photocurable artificial nail composition of the examples and the comparative examples.

Evaluation methods of the photocurable artificial nail compositions of the examples and the comparative examples are as follows. The evaluation was performed under indoor LED illumination at a room temperature of 23±2° C. and a humidity of 50±10% unless otherwise specified.

### [Measurement of Unpolymerization Ratio]

The photocurable artificial nail compositions of the examples and the comparative examples were each applied in a thickness of 0.2 mm on a glass flat plate, the weight was measured, and then the composition was cured by light irradiation with a commercially available gel nail light (PRESTO LED light manufactured by Nail Labo Corporation) for 20 seconds. The surface unpolymerized layer portion of the photocurable artificial nail composition was removed using a wipe soaked in ethanol, then the weight was measured again, and the unpolymerization ratio was calculated by Formula (2). The unpolymerization ratio of the photocurable artificial nail composition of the present invention is preferably 15.0 wt % or less, more preferably 13.5 wt % or less, still more preferably 11.0 wt % or less, and particularly preferably 9.0 wt % or less from the viewpoint of handleability, curability, and surface hardness.

$$Unpolymerization ratio ( wt \% ) = ( a - b ) / a \times 100 \qquad ( 2 )$$

wherein
a: weight of photocurable artificial nail composition before curing
b: weight of photocurable artificial nail composition after removal of surface unpolymerized layer

### [Curing Heat Measurement]

The photocurable artificial nail compositions of the examples and the comparative examples, 50 mg, were each uniformly applied to the region of Ø14 mm defined on a commercially available plastic plate (PRESTO clear pallet manufactured by Nail Labo Corporation). Thereafter, a thin temperature sensor (manufactured by RKC Instrument Inc., model: ST-50) connected to a portable thermometer (manufactured by RKC Instrument Inc., model: DP-700) was attached to the back part of the plastic plate, the plastic plate was irradiated with light from a commercially available gel nail light (PRESTO LED light manufactured by Nail Labo Corporation) for 20 seconds, and the temperature change of the photocurable artificial nail composition in the curing process was recorded. Here, the maximum temperature was defined as the curing heat, and the time from the start of light irradiation until the temperature reached the maximum temperature was defined as the time until reaching maximum temperature.

### [Yellowness Test]

The photocurable artificial nail compositions of the examples and the comparative examples were each filled in a spherical silicon mold of Φ12 mm, and cured by light irradiation with a commercially available gel nail light (PRESTO LED light manufactured by Nail Labo Corporation) for 20 seconds to prepare a spherical cured product.

The compositions before curing, immediately after curing, and at one day after curing were evaluated according to the following criteria.

### <Evaluation Criteria>

1: Strong coloring
2: Coloring is observed, but the composition is usable
3: Slight coloring is observed
4: Almost transparent without coloring
5: No coloring and transparent

### [Heat Pain Test]

A manicurist certified by the Japan Nailist Association, as a practitioner, conducted an operation for each of 10 subjects according to the following operation method, and carried out a heat pain test.

### <Operation Method>

Step 1: Sanding a natural nail surface with a sponge file.
Step 2: Wiping oil and dust with a wipe soaked in a nail cleanser.
Step 3: Applying a photocurable artificial nail composition of the examples or the comparative examples.
Step 4: Performing photopolymerization by light irradiation with commercially available gel nail light (PRESTO LED light manufactured by Nail Labo Corporation) for 20 seconds.

In the above operation steps, the heat pain felt by the subject during photopolymerization was sensory evaluated and rated according to the following criteria, and the average value of the evaluation values of 10 subjects (rounded off to the nearest whole number) was adopted.

### <Evaluation Criteria>

1: With significant heat pain
2: With slight heat pain
3: Feeling of curing heat but no heat pain
4: No feeling of curing heat

### [Tensile Test]

The photocurable artificial nail compositions of the examples and the comparative examples were each cured by light irradiation with a commercially available gel nail light (PRESTO LED light manufactured by Nail Labo Corporation) for 20 seconds to prepare a dumbbell-shaped test piece having a thickness of 1.9 mm, a total length of 28.6 mm, a distance between tabs of 25.0 mm, a length of the parallel part of 15.0 mm, a radius of the shoulder part of 7.0 mm, and a width of the parallel part of 2.0 mm, and the test piece was used as a test piece for measuring tensile strength. After standing all day and night, the tensile strength and breaking strain were measured using an Instron universal tester (manufactured by Instron Corporation, model: Instron 5943 type) under the condition of a crosshead speed of 10 mm/min.

### [Viscosity Measurement]

The photocurable artificial nail compositions of the examples and the comparative examples were each subjected to viscosity measurement under the following measurement conditions using a rheometer (manufactured by Anton Paar GmbH, model: Physica MCR 301), which is a dynamic viscoelasticity measuring device, and the measured value at a shear rate of 10 s⁻¹ was taken as the viscosity.

### (Measurement Conditions)

Measurement jig: Parallel plate PP20
Shear rate: 0.1 to 100 s⁻¹

TABLE 1

| | Example | Example | Example | Example | Example | Example | Example |
|---|---|---|---|---|---|---|---|
| Blended composition | 1 | 2 | 3 | 4 | 5 | 6 | 7 |
| A1 | 60 | 60 | 60 | 60 | 60 | 60 | 60 |
| A3 | 39.2 | 38.4 | 38 | 37.6 | 36.8 | 36 | 34 |
| Compound 1 | 0.8 | 1.6 | 2 | 2.4 | 3.2 | 4 | 6 |
| Total | 100 | 100 | 100 | 100 | 100 | 100 | 100 |
| Unpolymerization/wt % | 13.3 | 10.5 | 10.1 | 8.4 | 7.8 | 6.1 | 5.5 |
| Yellowness (visual) before curing | 5 | 5 | 4 | 4 | 4 | 4 | 4 |
| Yellowness (visual) immediately after | 5 | 5 | 4 | 4 | 4 | 4 | 4 |
| curing | | | | | | | |
| Yellowness (visual) at 1 day after curing | 5 | 5 | 4 | 4 | 4 | 4 | 4 |
| Curing heat/° C. | 45.4 | 46.1 | 45.1 | 45.1 | 46.1 | 46.9 | 48.0 |
| Time until reaching maximum | 16.0 | 14.7 | 14.2 | 13.8 | 13.5 | 13.0 | 13.5 |
| temperature/s | | | | | | | |
| Heat pain | 4 | 4 | 4 | 4 | 4 | 4 | 4 |
| Tensile strength/MPa | 26.2 | 30.1 | 28.1 | 26.3 | 26.3 | 26.3 | 26.9 |
| Breaking strain/% | 175.0 | 170.4 | 167.6 | 160.6 | 163.6 | 163.5 | 168.5 |
| Viscosity/Pas | 16.1 | 15.8 | 15.9 | 16.1 | 16.1 | 16.5 | 16.4 |

TABLE 2

| | | | Comparative | Comparative | Comparative | Example |
|---|---|---|---|---|---|---|
| Blended composition | Example 8 | Example 9 | Example 1 | Example 2 | Example 3 | 10 |
| A1 | 60 | 60 | 60 | 60 | 60 | 60 |
| A3 | 32 | 30 | 36 | 30 | 36 | 36 |
| Compound 1 | 8 | 10 | | | | 1 |
| Compound 2 | | | 4 | 10 | | 3 |
| Compound 3 | | | | | 4 | |
| Total | 100 | 100 | 100 | 100 | 100 | 100 |
| Unpolymerization/wt % | 5.4 | 4.6 | 5.7 | 4.7 | 5.0 | 6.2 |
| Yellowness (visual) before curing | 3 | 2 | 3 | 1 | 1 | 3 |
| Yellowness (visual) immediately after | 3 | 2 | 3 | 1 | 1 | 3 |
| curing | | | | | | |
| Yellowness (visual) at 1 day after curing | 3 | 2 | 3 | 1 | 1 | 4 |
| Curing heat/° C. | 46.3 | 46.4 | 45.9 | 47.5 | 44.2 | 46.6 |
| Time until reaching maximum | 13.0 | 12.7 | 10.3 | 8.7 | 8.5 | 11.3 |
| temperature/s | | | | | | |
| Heat pain | 4 | 4 | 3 | 2 | 2 | 3 |
| Tensile strength/MPa | 30.3 | 30.3 | 28.4 | 26.5 | 27.8 | 29.0 |
| Breaking strain/% | 171.1 | 161.4 | 174.2 | 176.4 | 165.0 | 171.0 |
| Viscosity/Pas | 17.5 | 17.4 | 15.6 | 18.4 | 16.6 | 15.2 |

TABLE 3

| | | | | Comparative |
|---|---|---|---|---|
| Blended composition | Example 11 | Example 12 | Example 13 | Example 4 |
| A1 | 60 | 60 | 48 | 48 |
| A2 | | | 20 | 20 |
| A3 | 36 | 36 | | |
| A4 | | | 15 | 15 |
| Compound 1 | 2 | 3 | 2 | |
| Compound 2 | 2 | 1 | | 2 |
| Pentaerythritol tetrakis(3-mercaptopropionate) | | | 15 | 15 |
| Total | 100 | 100 | 100 | 100 |
| Unpolymerization/wt % | 6.0 | 6.3 | 0.2 | 0.1 |
| Yellowness (visual) before curing | 3 | 4 | 3 | 1 |
| Yellowness (visual) immediately after curing | 3 | 4 | 4 | 2 |
| Yellowness (visual) at 1 day after curing | 4 | 4 | 4 | 2 |
| Curing heat/° C. | 47.2 | 47.6 | 73.1 | 82.4 |
| Time until reaching maximum temperature/s | 12.0 | 12.8 | 7.9 | 4.9 |
| Heat pain | 4 | 4 | 2 | 1 |
| Tensile strength/MPa | 31.0 | 28.6 | 40.2 | 42.3 |
| Breaking strain/% | 164.3 | 165.6 | 17.9 | 13.7 |
| Viscosity/Pas | 15.9 | 16.8 | 7.2 | 6.7 |

TABLE 4

| | Example | Comparative | Example | Comparative | Example | Comparative |
|---|---|---|---|---|---|---|
| Blended composition | 14 | Example 5 | 15 | Example 6 | 16 | Example 7 |
| A1 | 60 | 60 | 60 | 60 | 60 | 60 |
| A3 | 26 | 26 | 26 | 26 | 31 | 31 |
| Compound 1 | 4 | | 4 | | 4 | |
| Compound 2 | | 4 | | 4 | | 4 |
| Titanium oxide | | | | | 5 | 5 |
| Urethane beads | 10 | 10 | | | | |
| Acrylic beads | | | 10 | 10 | | |
| Total | 100 | 100 | 100 | 100 | 100 | 100 |
| Unpolymerization/wt % | 5.9 | 6.0 | 5.9 | 5.3 | 6.4 | 7.0 |
| Yellowness (visual) before | 4 | 3 | 4 | 3 | 4 | 3 |
| curing | | | | | | |
| Yellowness (visual) immediately | 4 | 3 | 4 | 3 | 3 | 2 |
| after curing | | | | | | |
| Yellowness (visual) at 1 day | 4 | 3 | 4 | 3 | 4 | 3 |
| after curing | | | | | | |
| Curing heat/° C. | 46.6 | 44.7 | 47.1 | 45.7 | 48.0 | 46.7 |
| Time until reaching maximum | 12.5 | 9.6 | 13.2 | 10.4 | 12.5 | 9.3 |
| temperature/s | | | | | | |
| Heat pain | 4 | 3 | 4 | 3 | 4 | 3 |
| Tensile strength/MPa | 29.8 | 30.9 | 25.6 | 25.7 | 25.0 | 30.2 |
| Breaking strain/% | 183.5 | 181.9 | 162.8 | 160.5 | 177.7 | 171.9 |
| Viscosity/Pas | 34.1 | 33.7 | 31 | 32.7 | 18.7 | 19.7 |

Urethane beads: particle diameter 6 μm (manufactured by Negami Chemical Industrial Co., Ltd., trade name: ART PEARL C-800)
Acrylic beads: particle diameter 8 μm (manufactured by Aica Kogyo Co., Ltd., trade name: GANZPEARL GMX-0810)

TABLE 5

| | | Comparative | | Comparative |
|---|---|---|---|---|
| Blended composition | Example 17 | Example 8 | Example 18 | Example 9 |
| A1 | 70 | 70 | 60 | 60 |
| A3 | 21 | 21 | 29 | 29 |
| A5 | | | 1 | 1 |
| A6 | | | 1 | 1 |
| A7 | | | 5 | 5 |
| Compound 1 | 4 | | 4 | |
| Compound 2 | | 4 | | 4 |
| Silica | 5 | 5 | | |
| Total | 100 | 100 | 100 | 100 |
| Unpolymerization/wt % | 4.8 | 5.0 | 4.5 | 4.6 |
| Yellowness (visual) before curing | 4 | 3 | 4 | 3 |
| Yellowness (visual) immediately after curing | 4 | 3 | 4 | 3 |
| Yellowness (visual) at 1 day after curing | 4 | 3 | 4 | 3 |
| Curing heat/° C. | 48.8 | 49.8 | 47.6 | 48.0 |
| Time until reaching maximum temperature/s | 13.4 | 9.3 | 13.4 | 10.7 |
| Heat pain | 4 | 3 | 4 | 3 |
| Tensile strength/MPa | 27.4 | 29.9 | 27.9 | 29.8 |
| Breaking strain/% | 177.9 | 170.5 | 137.0 | 132.9 |
| Viscosity/Pas | 77.3 | 79.8 | 27.8 | 24.7 |

Silica: manufactured by NIPPON AEROSIL Co.. Ltd., trade name: AEROSIL RX200)

According to the present invention, it is possible to provide a photocurable artificial nail composition that suppresses heat pain during curing, suppresses yellowing (including yellowing before curing), and has color tone stability.

1. A photocurable artificial nail composition comprising photopolymerization initiator and a polymerizable compound,
wherein the photopolymerization initiator contains a compound represented by General Formula (1), provided that the photopolymerization initiator does not contain an α-hydroxyalkylphenone photopolymerization initiator, and
the photocurable artificial nail composition does not contain a peroxide thermal polymerization initiator, 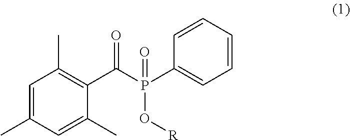
wherein R represents a hydrocarbon group having 1 to 5 carbon atoms.

2. The photocurable artificial nail composition according to claim 1, wherein the photopolymerization initiator contains only the compound represented by General Formula (1).

3. The photocurable artificial nail composition according to claim 1, wherein the photopolymerization initiator further contains 2,4,6-trimethylbenzoyldiphenylphosphine oxide and/or bis(2,4,6-trimethylbenzoyl)phenylphosphine oxide.

4. The photocurable artificial nail composition according to claim 1, wherein the polymerizable compound contains at least one selected from the group consisting of an oligomer and a polymerizable monomer.

5. The photocurable artificial nail composition according to claim 1, wherein the polymerizable compound contains an oligomer and a polymerizable monomer.

6. The photocurable artificial nail composition according to claim 4, wherein the oligomer contains a urethane di(meth)acrylate oligomer.

7. The photocurable artificial nail composition according to claim 1, containing 0.1 to 10 mass % of the compound represented by General Formula (1).

8. The photocurable artificial nail composition according to claim 1, containing 20 mass % or more of the polymerizable compound.

9. The photocurable artificial nail composition according to claim 5, wherein a content of the oligomer is 50 to 750 parts by mass per 100 parts by mass of the polymerizable monomer.

10. The photocurable artificial nail composition according to claim 1 which has a viscosity of 1 to 2,000,000 mPa·s.

11. An artificial nail obtained from the photocurable artificial nail composition according to claim 1.

12. A method for producing an artificial nail, comprising the step of applying the photocurable artificial nail composition according to claim 1 onto a nail or a substrate and curing the photocurable artificial nail composition.

13. The photocurable artificial nail composition according to claim 5, wherein the oligomer contains a urethane di(meth)acrylate oligomer.
